(19) United States
(12) Patent Application Publication
(10) Pub. No.: US 2005/0186270 A1
(43) Pub. Date: Aug. 25, 2005

(54) Stable tablets comprising simvastatin

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 10/510,474

(22) PCT Filed: Apr. 3, 2003
(86) PCT No.: PCT/CA03/00479

(30) Foreign Application Priority Data
Apr. 9, 2002 (CA) 2379887

(51) Int. Cl. A61K 9/20; A61K 31/225

(52) U.S. Cl. 424/464; 514/548

(57) ABSTRACT

A tablet comprising simvastatin and excipients, wherein the content of lactose, if any, is less than 75 percent of the total excipients by weight, and wherein the content of cellulose is more than 20 percent of the total excipients by weight.

### BACKGROUND OF THE INVENTION

Simvastatin is a lipid-lowering agent that is produced synthetically from lovastatin. Simvastatin is disclosed and claimed in U.S. Pat. No. 4,444,784. Tablets comprising simvastatin as the active ingredient are sold by Merck & Co., Inc. in the United States and elsewhere under the tradename Zocor™ in strengths of 5 mg, 10 mg, 20 mg, 40 mg and 80 mg.

With respect to pharmaceutical formulations (i.e. dosage forms) comprising simvastatin, the only information disclosed in U.S. Pat. No. 4,444,784 is a statement that typical formulations for filling hard gelatin capsules comprise the active drug and finely divided lactose.

Zocor™ tablets are film-coated tablets, by which is meant that they consist of core tablets surrounded by a water-soluble film coating. The labelling for Zocor™ tablets indicate that the excipients (i.e. inactive ingredients) used in the core tablets are lactose, cellulose, starch, magnesium stearate, ascorbic acid, citric acid and butylated hydroxyanisole (also known as BHA).

Lactose, cellulose, starch, and magnesium stearate are all very commonly used as excipients in tablets. In particular, lactose and cellulose are very commonly used as fillers and binders. When lactose is used as an excipient, it is typically used in the largest quantity and typically constitutes more than 75 percent of the total excipients by weight. Starch is very commonly used as a filler and disintegrant, and magnesium stearate is very commonly used as a lubricant, to avoid sticking and binding in the tabletting process.

The inclusion of BHA, ascorbic acid, and citric acid as excipients in the core tablets is unusual, and the inclusion of these excipients indicates that Merck & Co. Inc. found that it was necessary to include these excipients to achieve satisfactory stability of the simvastatin in the tablet. Simvastatin is prone to degradation due to oxidation of the diene and oxidation of the hydroxyl group in the simvastatin molecule. BHA and ascorbic acid are apparently included in the tablets as antioxidants. Citric acid is apparently added because it has chelation properties with metal ions, which, in the absence of the citric acid, could catalyze the oxidation process.

The composition of the Zocor™ core tablets is thus relatively complex in terms of the number of excipients used. Also, the use of BHA as an antioxidant usually requires the use of a solvent. That is to say, BHA is dissolved in a solvent, the solution is used to granulate the simvastatin, optionally after mixing with one or more excipients, and the wet mass is then dried to evaporate the solvent. The process of manufacture of the tablets is thus much more complex and expensive than a simple dry-mix process, by which is meant a process in which all of the ingredients are mixed together in dry form, and the mixture is compressed into tablets, without adding a solvent and then drying to evaporate the solvent. It is clearly preferable to avoid the use of solvents, if possible, in order to simplify the process of manufacture.

Simvastatin is also a compound for which it is difficult to produce a tablet formulation which exhibits rapid absorption after ingestion. It is necessary that any tablet formulation that is developed as an alternative to Zocor™ exhibit a rate of absorption on oral administration that is equivalent to that of Zocor™.

In light of this prior art, the objective of the present invention is to enable the manufacture of simvastatin tablets so as to achieve at least one or more, if not all, of the following:
1. Improved stability relative to tablets that use lactose as the predominant excipient.
2. Rate and extent of absorption equivalent to Zocor™ upon oral administration.
3. Elimination of the need to include citric acid as an excipient.
4. Elimination of the need to include ascorbic acid as an excipient.
5. Elimination of the need to include BHA as an excipient.
6. Production by a dry-mix method; i.e. without the need to granulate with a solvent and then dry to evaporate the solvent.

### BRIEF SUMMARY OF THE INVENTION

It has been found that rate of degradation of simvastatin is significantly affected by the excipients with which it is mixed.

More particularly, it has been found that the stability of simvastatin in tablets is significantly improved by reducing or eliminating the lactose content, and by using cellulose, as a major excipient. The tablets of the present invention thus comprise simvastatin and excipients, wherein the content of lactose, if any, is less than 75 percent of the total excipients by weight, and wherein the content of cellulose is more than 20 percent of the total excipients by weight.

### DETAILED DESCRIPTION OF THE INVENTION

Tablets comprising simvastatin will generally be made by mixing simvastatin with excipients (inactive ingredients) and compressing the mixture into tablets on a tablet press.

Among ingredients most commonly used as fillers and binders in pharmaceutical tablets are lactose (which may be either anhydrous lactose or lactose monohydrate) and cellulose. They are considered to be binders as well as fillers, because they usually enable compression into hard tablets, if they are the predominant ingredients.

Because lactose is the predominant excipient used in Zocor™ tablets, which presumably were carefully developed to Merck & Co. Inc. for maximum stability, it has been surprising to discover that stability is better with cellulose than with lactose as principal excipient.

Tablets of the present invention will have a lactose content that is less than 75 percent, is preferably less than 60 percent, and is more preferably less than 40 percent of the total excipient content by weight. Most preferably the tablets will be lactose free.

The tablets will comprise cellulose (which may be either microcrystalline cellulose or powdered cellulose) as a filler and binder. The amount of cellulose will exceed 20 percent, will preferably exceed 40 percent, and will more preferably exceed 60 percent of the total excipients by weight.

Cellulose is often considered to be a disintegrant in addition to being a filler and binder, because, like other disintegrants, it absorbs water and swells, thus aiding in the disintegration of tablets containing cellulose when they are added to an aqueous medium. Nevertheless, tablets of the present invention will preferably also comprise a disintegrant other than cellulose.

Commonly used disintegrants include starch (which may be pregelatinized), croscarmellose sodium, carmellose calcium, sodium starch glycolate, and crospovidone. It has been further found that stability of the simvastatin tablets is better when starch, sodium starch glycolate, or crospovidone is used as disintegrant, than when either croscarmellose sodium or carmellose calcium is used. Accordingly, the tablets of the present invention will preferably comprise starch, sodium starch glycolate or crospovidone, and will preferably not comprise any croscarmellose sodium or carmellose calcium.

The presence of an adequate amount of a disintegrant other than cellulose is necessary to ensure that the tablets are bioequivalent to Zocor™; that is to say that the rate of absorption of the simvastatin is equivalent to that of Zocor™ upon ingestion.

The United States Pharmacopoeia, 25^{th} edition, includes a dissolution test for simvastatin tablets. The test is done in apparatus 2 at 50 rpm in 900 mL of pH 7.0 buffer solution containing 0.5 percent dodecyl sodium sulfate in 0.01M sodium phosphate. The specification requires dissolution of not less than 75 percent in 30 minutes.

While tablets which comprise cellulose as the predominant excipient and which do not also comprise another disintegrant may exhibit rapid disintegration in water and may even pass the United States Pharmacopoeia dissolution test, such tablets will nevertheless be unlikely to be bioequivalent to Zocor™ tablets, as a result of exhibiting slower dissolution than Zocor™ in gastric fluid, which has a lower pH than used in the United States Pharmacopoeia test.

Where starch is used as the disintegrant, the quantity will preferably exceed 12 percent and more preferably exceed 20 percent of the total of excipients by weight. Where the disintegrant is selected from sodium starch glycolate, crospovidone, croscarmellose sodium or carmellose calcium (of which the first two of these are preferable as aforesaid for reasons of stability), the amount will preferably exceed 1 percent, more preferably exceed 2 percent, and even more preferably exceed 3 percent of the total of excipients by weight.

The total of excipients selected from the group consisting of cellulose, starch, sodium starch glycolate will preferably exceed 65 percent, will more preferably exceed 80 percent, will even more preferably exceed 90 percent, and will most preferably exceed 95 percent of the total of excipients by weight.

It has been found that the stability of simvastatin in tablets is also significantly affected by the choice of lubricant. Stability is improved when magnesium stearate, which is by far the most commonly used lubricant, is replaced by zinc stearate, or sodium stearyl fumarate. Thus tablets of this present invention will preferably be free of magnesium stearate, and will preferably comprise zinc stearate or sodium stearyl fumarate.

By selecting excipients in accordance with the teaching of this disclosure, it is possible to significantly improve the stability of simvastatin so as to reduce or eliminate the need for the stabilizers that are included in Zocor™ tablets.

Tablets of the present invention will thus optionally and preferably be free of citric acid. The tablets will also optionally and preferably be free of ascorbic acid. The tablets will preferably be free of both citric and ascorbic acid. The tablets will also optionally and preferably be free of BHA. The tablets will preferably be free of all three of citric acid, ascorbic acid and BHA.

As aforesaid, the inclusion of BHA in Zocor™ tablets requires the use of a wet-granulation process in which the BHA is dissolved in solvent, the solution is used to wet granulate the simvastatin (after it is mixed with excipients), and the wet mass is then dried to evaporate the solvent.

The tablets of the present invention will preferably be made by a dry-mix process; that is to say, a process in which all of the ingredients are mixed together in dry form, without any step of adding solvent and then drying to evaporate the solvent.

The invention will be better understood from the following examples, which are meant to be illustrative, and not limiting of the scope of the invention.

### EXAMPLES 1 TO 7

| | Example No. | | | | | | |
|---|---|---|---|---|---|---|---|
| | 1 | 2 | 3 | 4 | 5 | 6 | 7 |
| Simvastatin | 5.0 | 5.0 | 5.0 | 5.0 | 5.0 | 5.0 | 5.0 |
| Anhydrous Lactose | 39.6 | 25.0 | 25.0 | 25.0 | 25.0 | 25.0 | 25.0 |
| Microcrystalline | 0 | 14.6 | 0 | 0 | 0 | 0 | 0 |
| Cellulose | | | | | | | |
| Starch, Pregelatinized | 0 | 0 | 14.6 | 0 | 0 | 0 | 0 |
| Croscarmellose | 0 | 0 | 0 | 14.6 | 0 | 0 | 0 |
| Sodium | | | | | | | |
| Carmellose Calcium | 0 | 0 | 0 | 0 | 14.6 | 0 | 0 |
| Sodium Starch | 0 | 0 | 0 | 0 | 0 | 14.6 | 0 |
| Glycolate | | | | | | | |
| Crospovidone | 0 | 0 | 0 | 0 | 0 | 0 | 14.6 |
| Magnesium Stearate | 0.4 | 0.4 | 0.4 | 0.4 | 0.4 | 0.4 | 0.4 |
| | 45.0 | 45.0 | 45.0 | 45.0 | 45.0 | 45.0 | 45.0 |

For each of examples 1 to 7, the ingredients were mixed in the proportions shown, and the powder mixture was then compressed into tablets of unit weight 45 mg, so that each tablet comprised about 5 mg of simvastatin. In the tablets of example 1, the only excipients are lactose as filler-binder, and magnesium stearate as lubricant. In each of examples 2 to 7, 14.6 mg of the lactose is replaced by 14.6 mg of one of cellulose, starch, croscarmellose sodium, carmellose calcium, sodium starch glycolate and crospovidone.

Tablets of each of examples 1 to 7 were stored at 60° C. for four weeks and then tested for the amount of simvastatin-oxolactone, which is the product of oxidation of the hydroxyl group in the simvastatin molecule. The testing was done by an HPLC (high performance liquid chromatographic) method, with results as follows, as a percentage of the simvastatin content:

| | Example No. | | | | | | |
|---|---|---|---|---|---|---|---|
| | 1 | 2 | 3 | 4 | 5 | 6 | 7 |
| % Oxolactone | .108% | .104% | .154% | .169% | .187% | .142% | .118% |

The initial level of oxolactone in the simvastatin used to make the tablets was about 0.04%.

Comparing the results for examples 1 and 2 indicates that replacing part of the lactose by cellulose reduced the rate of oxidation.

Comparing the results of examples 3 to 7 with the result of example 1 indicates that the addition of any of the five disintegrants increases the rate of oxidation (at least when lactose is still the primary excipient), but that the rate of oxidation is lower when the disintegrant is crospovidone, sodium starch glycolate, or starch, than when it is croscarmellose sodium or carmellose calcium.

### EXAMPLES 8 TO 13

| | Example No.: | | | | | |
|---|---|---|---|---|---|---|
| | 8 | 9 | 10 | 11 | 12 | 13 |
| Simvastatin | 5.0 | 5.0 | 5.0 | 5.0 | 5.0 | 5.0 |
| Microcrystalline Cellulose | 39.9 | 25.3 | 25.3 | 25.3 | 25.3 | 25.3 |
| Starch, Pregelatanized | 0 | 14.6 | 0 | 0 | 0 | 0 |
| Croscarmellose Sodium | 0 | 0 | 14.6 | 0 | 0 | 0 |
| Carmellose Calcium | 0 | 0 | 0 | 14.6 | 0 | 0 |
| Sodium Starch Glycolate | 0 | 0 | 0 | 0 | 14.6 | 0 |
| Crospovidone | 0 | 0 | 0 | 0 | 0 | 14.6 |
| Magnesium Stearate | 0.1 | 0.1 | 0.1 | 0.1 | 0.1 | 0.1 |
| | 45.0 | 45.0 | 45.0 | 45.0 | 45.0 | 45.0 |

Examples 8 to 13 are repeats of examples 2 to 7, with all of the lactose replaced by cellulose, and the amount of magnesium stearate per tablet reduced from 0.4 mg to 0.1 mg. The tablets were made by the same process as used in examples 1 to 7.

Sample tablets were also stored at 60° C. for 4 weeks and tested by the same method, with results as follows:

| | Example No. | | | | | |
|---|---|---|---|---|---|---|
| | 8. | 9 | 10 | 11 | 12 | 13 |
| % Oxolactone | .098% | .113% | .129% | .143% | .101% | .095% |

Comparing the result for example 8 to the results for examples 1 and 2 confirms that the rate of oxidation is further reduced by replacing the balance of the lactose with more cellulose. Comparing the results of examples 9 to 13 again confirms that the rate of oxidation is lower when the disintegrant is one of crospovidone, sodium starch glycolate or starch, than when it is croscarmellose sodium or carmellose calcium.

### EXAMPLES 14 AND 15

Example No.

| | 14 | 15 |
|---|---|---|
| Simvastatin | 5.0 | 5.0 |
| Crospovidone | 34.9 | 34.9 |
| Magnesium Stearate | 0.1 | 0 |
| Zinc Stearate | 0 | 0.1 |
| | 40.0 | 40.0 |

Tablets of examples 14 and 15 were made by the same process as examples 1 to 13, except that the tablets were made at a unit weight of 40 mg instead of 45 mg. The purpose of examples 14 and 15 was to compare the effect on oxidation rate when magnesium stearate is replaced by zinc stearate as lubricant.

Again tablets were stored at 60° C., and after 4 weeks samples were tested with results as follows:

Example No.

| | 14 | 15 |
|---|---|---|
| % Oxolactone | 0.193% | 0.146% |

These results confirm that the oxidation rate is reduced by eliminating magnesium stearate as lubricant, and replacing it by zinc stearate. Comparing the results of examples 14 and 13, also shows that the oxidation rate is lower when the excipient content is mostly cellulose than when it is mostly crospovidone.

### EXAMPLES 16 TO 21

| | Example No. | | | | | |
|---|---|---|---|---|---|---|
| | 16 | 17 | 18 | 19 | 20 | 21 |
| Simvastatin | 5.0 | 5.0 | 5.0 | 5.0 | 5.0 | 5.0 |
| Microcrystalline Cellulose | 39.9 | 30.0 | 39.9 | 30.0 | 40.0 | 30.0 |
| Crospovidone | 0 | 9.9 | 0 | 9.9 | 0 | 10.0 |
| Zinc Stearate | 0.1 | 0.1 | 0 | 0 | 0 | 0 |
| Sodium Stearyl Fumarate | 0 | 0 | 0.1 | 0.1 | 0 | 0 |
| | 45.0 | 45.0 | 45.0 | 45.0 | 45.0 | 45.0 |

Tablets of examples 16 to 21 were made by the same process as examples 1 to 13. The purpose of these examples was to compare the stability of tablets with zinc stearate as lubricant, sodium stearyl fumarate as lubricant, and no lubricant at all, all both with and without crospovidone as disintegrant.

Tablets for each of examples 16 to 21 were stored at 60° C. for 2 weeks, and then tested with results as follows:

| | Example No. | | | | | |
|---|---|---|---|---|---|---|
| | 16 | 17 | 18 | 19 | 20 | 21 |
| % Oxolactone | .055% | .055% | .046% | .051% | .046% | .041% |

These results indicate good stability, regardless of whether the tablets contain sodium stearyl fumarate or zinc stearate as lubricant, or no lubricant at all.

1. A pharmaceutical tablet which comprises simvastatin and excipients, wherein the amount of lactose, if any, is less than 75 percent of the total of excipients by weight, and wherein the amount of cellulose is more than 20 percent of the total excipients by weight.

2. The tablet of claim 1 wherein the amount of lactose is less than 60 percent of the total of excipients by weight.

3. The tablet of claim 1 wherein the amount of lactose is less than 40 percent of the total of excipients by weight.

4. The tablet of claim 1 that is lactose free.

5. The tablet of any of claim 1 wherein the amount of cellulose is more than 40 percent of the total of excipients by weight.

6. The tablet of claim 1 wherein the amount of cellulose is more than 60 percent of the total of excipients by weight.

7. The tablet of claim 1 that comprises as a disintegrant other than cellulose.

8. The tablet of claim 7 wherein the disintegrant is starch.

9. The tablet of claim 8 wherein the amount of starch exceeds 12 percent of the total of excipients by weight.

10. The tablet of claim 8 wherein the amount of starch exceeds 20 percent of the total of excipients by weight.

11. The tablet of claim 7 wherein the disintegrant is crospovidone.

12. The tablet of claim 7 wherein the disintegrant is sodium starch glycolate.

13. The tablet of claim 7 wherein the disintegrant is either croscarmellose sodium or carmellose calcium.

14. The tablet of any one of claims 11 to 13 wherein the amount of the disintegrant exceeds 1 percent of the total of excipients by weight.

15. The tablet of any one of claims 1 1 to 13 wherein the amount of the disintegrant exceeds 2 percent of the total of excipients by weight.

16. The tablet of any one of claims 11 to 13 wherein the amount of the disintegrant exceeds 3 percent of the total of excipients by weight.

17. The tablet of claim 1 wherein excipients selected from the group consisting of cellulose, starch, sodium starch glycolate, and crospovidone exceed 65 percent of the total excipients by weight.

18. The tablet of claim 1 wherein excipients selected from the group consisting of cellulose, starch, sodium starch glycolate, and crospovidone exceed 80 percent of the total excipients by weight.

19. The tablet of claim 1 wherein excipients selected from the group consisting of cellulose, starch, sodium starch glycolate, and crospovidone exceed 90 percent of the total excipients by weight.

20. The tablet of claim 1 wherein excipients selected from the group consisting of cellulose, starch, sodium starch glycolate, and crospovidone exceed 95 percent of the total excipients by weight.

21. The tablet of claim 1 that is free of magnesium stearate.

22. The tablet of claim 1 that comprises zinc stearate.

23. The tablet of claim 1 that comprises sodium stearyl fumarate.

24. The tablet of claim 1 that is free of citric acid.

25. The tablet of claim 1 that is free of ascorbic acid.

26. The tablet of claim 1 that is free of butylated hydroxyanisole.

27. The tablet of claim 1 when made by a dry-mix process.
